(19) United States
(12) Patent Application Publication
(10) Pub. No.: US 2026/0077023 A1
(43) Pub. Date: Mar. 19, 2026

(54) ANTIMICROBIAL HYDROLYZED COLLAGEN WITH POLYLYSINE AND METHODS OF USING THE SAME

(71) Applicant: Rochal Technologies, LLC, San Antonio, TX (US)

(72) Inventors: Bradley RODIER, San Antonio, TX (US); Larry ESTLACK, San Antonio, TX (US); Rebecca McMAHON, San Antonio, TX (US); Nina BIONDA, Ithaca, NY (US); Ann BEAL SALAMONE, Gainesville, FL (US); Tiffany Fawn SANCHEZ, Helotes, TX (US); Sergio MEDINA, San Antonio, TX (US); Jonathan VELASCO, San Antonio, TX (US); Fahimeh TABATABAEI, Boerne, TX (US)

(73) Assignee: Rochal Technologies, LLC, San Antonio, TX (US)

(21) Appl. No.: 19/328,859

(22) Filed: Sep. 15, 2025

Related U.S. Application Data
(63) Continuation of application No. PCT/US2025/046116, filed on Sep. 12, 2025.
(60) Provisional application No. 63/694,835, filed on Sep. 14, 2024.

(51) Int. Cl. A61K 38/39; A61K 47/34; A61P 31/04

(52) U.S. Cl. CPC A61K 38/39; A61K 47/34; A61P 31/04

(57) ABSTRACT

A hydrolyzed collagen-based composition comprising hydrolyzed collagen and 0.01 to 50 wt-% of polylysine based on the percent solids of the hydrolyzed collagen-based composition is provided. Methods of treating tissue (e.g., impaired tissue) are also provided.

### CROSS-REFERENCE TO RELATED APPLICATIONS

This application is a continuation of International Application No. PCT/US2025/046116, filed Sep. 12, 2025, and claims priority under 35 U.S.C. 119(e) to U.S. Application No. 63/694,835, filed on Sep. 14, 2024, the entirety of which is incorporated herein by reference.

### FIELD OF INVENTION

A hydrolyzed collagen composition with biocidal activity is described, in particular a composition with hydrolyzed collagen and polylysine that reduces or eliminates microbial infections.

### BACKGROUND

Hydrolyzed collagen is derived from acid, alkaline, or enzymatic hydrolysis of collagen (molecular weight of about 300 kDa) to a molecular weight of generally less than 10 kDa. Hydrolyzed collagens have high concentrations of oligomers containing hydroxyproline (hydrophilic), proline (hydrophobic) and glycine (hydrophilic), low content of sulfur-containing amino acids, and no tryptophan.

When dissolved in aqueous media, hydrolyzed collagen produces a low viscosity solution at lower concentrations, but can form a water-soluble gel at higher concentrations (e.g., over 60 wt-%). When dried, this hydrolyzed collagen gel forms a brittle coating that is readily water soluble.

Polylysine is found in nature as ε-poly-L-lysine; while α-polylysine can be synthesized through condensation polymerization of lysine.

### SUMMARY OF THE INVENTION

In some aspects, a hydrolyzed collagen-based composition comprising hydrolyzed collagen and 0.01 to 50 wt-% of polylysine based on the percent solids of the composition is provided. The hydrolyzed collagen-based composition can have antimicrobial properties to mitigate or eliminate infection while maintaining or improving biocompatibility. In some embodiments, the hydrolyzed collagen-based composition has a selectivity index of at least 2 against at least one planktonic microbe.

In some embodiments, the hydrolyzed collagen-based compositions described herein can be used to reduce or eliminate microbes in food and nutritional supplements.

In some embodiments, the hydrolyzed collagen-based compositions described herein can be used in cosmetics and personal care products.

In some embodiments, the hydrolyzed collagen-based compositions described herein can be used to treat surgical incision sites.

In some embodiments, the hydrolyzed collagen-based compositions described herein can be used to treat impaired soft tissue and hard tissue.

In some embodiments, the hydrolyzed collagen-based compositions described herein can be used to treat acute and chronic wounds, as well as burn wounds.

In some embodiments, the hydrolyzed collagen-based compositions described herein can be used to reduce and eliminate Gram-positive and Gram-negative bacteria in wounds, tissues, surfaces and devices.

In some embodiments, the hydrolyzed collagen-based compositions described herein can be used to reduce and eliminate fungi in wounds, tissues, surfaces and devices.

In some embodiments, the hydrolyzed collagen-based compositions described herein can be used to reduce and eliminate yeast in wounds, tissues, surfaces and devices.

In some embodiments, the hydrolyzed collagen-based compositions described herein can be used to reduce and eliminate mold in wounds, tissues, surfaces and devices.

In some embodiments, the hydrolyzed collagen-based compositions described herein can be used to reduce and eliminate viruses in wounds, tissues, surfaces and devices.

In some embodiments, the hydrolyzed collagen-based compositions described herein can be used to facilitate tissue healing by incorporation of hydrolyzed collagen and polylysine.

In some embodiments, the hydrolyzed collagen-based compositions described herein can provide polylysine-containing compositions that are non-cytotoxic to mammalian cells.

In some embodiments, the hydrolyzed collagen-based compositions described herein can be deposited on a surface or a device to give enhanced or total elimination of microbial activity and biocompatibility.

These and other objectives and advantages of the hydrolyzed collagen-based compositions described herein, some of which are specifically described and others that are not, will become apparent from the detailed description and drawings that follow.

### BRIEF DESCRIPTION OF DRAWINGS

FIG. 1 shows MTT data for hydrolyzed collagen with and without polylysine.

### DETAILED DESCRIPTION OF THE INVENTION

In some aspects, a hydrolyzed collagen-based composition is provided that includes hydrolyzed collagen and 0.01 to 50 wt-% of polylysine based on the percent solids of the hydrolyzed collagen-based composition. As used herein, “percent solids” has its standard meaning of the quantification of the non-volatile ingredients in a composition.

In some embodiments, the hydrolyzed collagen-based composition comprises 50 to 99.99 wt-% of hydrolyzed collagen. In some embodiments, the hydrolyzed collagen-based composition comprises up to 99.925 wt-% or up to 99.9 wt-% of hydrolyzed collagen based on the percentage of solids in the composition. In some embodiments, the hydrolyzed collagen-based composition comprises at least 50 wt-%, at least 60 wt-%, at least 70 wt-%, at least 80 wt-%, or at least 90 wt-% of hydrolyzed collagen based on the percentage of solids in the composition.

In some embodiments, the source of collagen used to form the hydrolyzed collagen can be human, bovine, porcine, piscine, ovine, poultry, or another source. In some embodiments, the hydrolyzed collagen is derived from bovine collagen.

In some embodiments, the hydrolyzed collagen-based composition comprises 0.01 to 50 wt-% of polylysine based on the percent solids of the composition. In some embodiments, the hydrolyzed collagen-based composition comprises 0.05 to 25 wt-% or 0.1 to 2 wt-% of polylysine based on the percent solids of the composition. In some embodiments, the hydrolyzed collagen-based composition comprises up to 40 wt-%, up to 30 wt-%, up to 20 wt-%, or 15 wt-%, or 10 wt-%, or 5 wt-%, or 3 wt-% of polylysine based on the percent solids of the composition.

Polylysine may be present naturally or can be derived synthetically. Polylysine is a biocompatible, safe polymer, which can have microbiostatic properties. The presence of high protein levels does not affect polylysine efficacy, which is an advantage in the hydrolyzed collagen-based compositions described herein as well as during treatment. In the hydrolyzed collagen-based compositions described herein, all polylysines with microbiostatic efficacy can be used alone or in combination thereof. Polylysine exists as both α-polylysine and ε-polylysine.

ε-Poly-L-lysine (ε-PLL) is a naturally occurring, cationic homopoly(amino acid) composed of 25 to 35 repeat units of L-lysine (MW 3.2-4.5 kD) in a β-sheet conformation with a degree of crystallinity estimated at 63% (Maeda, 2003). Computational studies of polylysine provide further understanding that cyclohepta bifurcated hydrogen bonds between the β-sheet folded ε-poly-L-lysine segments are a key differentiator between ε-poly-L-lysine and α-poly-L-lysine, which may contribute to ε-poly-L-lysine's higher antimicrobial activity (Jia, 2009). Studies with Gram-negative E. coli have determined that ε-poly-L-lysine not only adsorbs onto the cell surface but disrupts the outer membrane and affects cytoplasmic metabolism resulting in cell respiration decreases, DNA and RNA biosynthesis decreases, and cell wall biosynthesis decreases (Shima, 1984). Similarly, mechanistic studies with Gram-positive S. aureus in the presence of polylysine demonstrate attachment of the polylysine to the bacterial outer cell wall. This attachment is created by interaction between the negatively charged teichoic acid in the peptidoglycan cell wall and the cationic polylysine. Peptidoglycan (glycosaminoglycan chains crosslinked by short peptides) is a key structural component of a bacterial cell wall. Thus, the outer cell wall is disrupted, and ε-poly-L-lysine then interacts with the cell (plasma) membrane and increases its permeability. Polylysine entrance into the cytoplasm inhibits glycolysis and subsequent energy generation needed to create new intracellular and extracellular construction amino acids, carbohydrates, etc. (Tan, 2019). ε-Poly-L-lysine is principally used as a food preservative due to its biocompatibility.

In some embodiments, the hydrolyzed collagen-based composition has a selectivity index of at least 2 against at least one planktonic microbe relative to mammalian cell toxicity. In some embodiments, the hydrolyzed collagen-based composition has a selectivity index of at least 5 or at least 7 or at least 10.

In some embodiments, the at least one planktonic microbe is a Gram-negative bacteria, a Gram-positive bacteria, a fungi, or a virus. In some embodiments, the selectivity index can be based on any planktonic microbe, including but not limited to the planktonic microbes disclosed herein. In some embodiments, the mammalian cell is fibroblasts, keratinocytes, endothelial cells, macrophages, mesenchymal stem cells, osteoblasts, osteocytes, or osteoclasts. In some embodiments, the mammalian cell is fibroblasts.

As used herein, “selectivity index” has its ordinary meaning. For example, the selectivity index can be an index equal to the concentration of a composition needed to kill >90% of a mammalian cell (e.g., fibroblasts) in a standard 2D in vitro cell culture assay, divided by the concentration of a composition needed to kill at least 6 logs of a planktonic microbe in the same standard 2D in vitro cell culture assay.

Polylysine can also be α-polylysine, which is a manmade polymer that generally has a molecular weight ranging from 15,000 to 70,000 Da.

The α-poly-D-lysine component of α-polylysine is resistant to biodegradation and, thus, serves as a substrate for cell attachment for a prolonged period compared to α-poly-L-lysine. Thus, while ε-polylysine is preferred for antimicrobial activity, α-polylysine is useful for cell viability.

In some embodiments, the polylysine comprises ε-polylysine. In some embodiments, the polylysine comprises ε-poly-L-lysine (ε-PLL).

In some embodiments, the polylysine comprises α-polylysine. In some embodiments, the polylysine comprises α-poly-L-lysine ((α-PLL). In some embodiments, the polylysine comprises α-poly-D-lysine (α-PDL).

In some embodiments, the polylysine comprises both ε-polylysine and α-polylysine. In some embodiments, the polylysine includes ε-PLL and α-PDL.

In some embodiments, a weight ratio of ε-polylysine to α-polylysine ranges from 1:5 to 5:1. In some embodiments, a ratio of ε-polylysine to α-polylysine ranges from 1:4 to 4:1, or from 1:3 to 3:1, or from 2:1 to 1:2.

In some embodiments, a weight ratio of ε-PLL to α-PDL ranges from 1:5 to 5:1. In some embodiments, a ratio of ε-polylysine to α-polylysine ranges from 1:4 to 4:1, or from 1:3 to 3:1, or from 2:1 to 1:2.

In some embodiments, the polylysine comprises more ε-polylysine than α-polylysine.

In some embodiments, the polylysine comprises more α-polylysine than ε-polylysine.

In some embodiments, the hydrolyzed collagen-based composition comprises 0.01 to 50 wt-% of ε-polylysine based on the percent solids of the composition. In some embodiments, the hydrolyzed collagen-based composition comprises 0.05 to 25 wt-% or 0.1 to 2 wt-% of ε-polylysine based on the percent solids of the composition. In some embodiments, the hydrolyzed collagen-based composition comprises up to 40 wt-%, up to 30 wt-%, up to 20 wt-%, or 15 wt-%, or 10 wt-%, or 5 wt-%, or 3 wt-% of ε-polylysine based on the percent solids of the composition.

In some embodiments, the hydrolyzed collagen-based composition comprises 0.01 to 50 wt-% of α-polylysine based on the percent solids of the composition. In some embodiments, the hydrolyzed collagen-based composition comprises 0.05 to 25 wt-% or 0.1 to 2 wt-% of α-polylysine based on the percent solids of the composition. In some embodiments, the hydrolyzed collagen-based composition comprises up to 40 wt-%, up to 30 wt-%, up to 20 wt-%, or 15 wt-%, or 10 wt-%, or 5 wt-%, or 3 wt-% of α-polylysine based on the percent solids of the composition.

In some embodiments, the hydrolyzed collagen-based composition exhibits a kill rate of at least 1 log reduction of a planktonic microbe. In some embodiments, the composition exhibits a kill rate of at least 2 log reduction, or at least 3 log reduction, or at least 4 log reduction, or at least 5 log reduction of a planktonic microbe. In some embodiments, the planktonic microbe is at least one of Gram positive bacteria, Gram negative bacteria, fungi, yeast. or virus. Examples of specific planktonic microbes include, but are not limited to, P. aeruginosa, C. albicans, are methicillin-resistant S. aureus (MRSA).

In some embodiments, the hydrolyzed collagen-based composition enhances a natural tissue regeneration process. In some embodiments, the natural tissue regeneration process that is enhanced is at least one of cell migration, cell proliferation, cell viability, cell differentiation, and angiogenesis. In some embodiments, the cells are fibroblasts. As used herein, a natural tissue regeneration process is enhanced if the property is improved compared to an untreated control. In some embodiments, at least one natural tissue regeneration process is improved by at least 5%., or at least 10%, or at least 15%, or at least 20% compared to an untreated control.

In some embodiments, the hydrolyzed collagen-based composition comprises 0.1 to 5 wt-% of a laurate ester based on the percent solids of the composition. In some embodiments, the laurate ester is monolaurin.

In some embodiments, the hydrolyzed collagen-based composition comprises a chelating agent selected from the group consisting of aminocarboxylic acids, citric acid and its salts, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N-bis(2-hydroxybenzyl)ethylenediamine-N,N-diacetic acid, ethylenediamine-N,N-diacetic acid, ethylenediamine-N,N-dipropionic acid, triethylenetetraaminehexaacetic acid, ethylenediamine-N,N′-bis(methylenephosphonic acid), iminodiacetic acid, N,N-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates. In some embodiments, the hydrolyzed collagen-based composition comprises EDTA.

In some embodiments, the hydrolyzed collagen-based composition may comprise a chelating agent in an amount of 0.01 wt % to 5 wt % or 0.05 wt % to 3 wt % based on the percent solids of the composition.

In some embodiments, the hydrolyzed collagen-based composition has a form selected from a powder, a liquid, a gel, a paste, a cream, a suspension, an emulsion, a film, a sheet, a foam, a lotion, a spray, an aerosol, a capsule, or a tablet. In some embodiments, the composition is a powder.

In some embodiments, the polylysine may be incorporated in the hydrolyzed collagen-based composition using a variety of methods such as blending the polylysine as a powder or as a solution, using a solvent such as aqueous media. If a solution is used, the solvent can be evaporated or can remain in the final form.

In some embodiments, polylysine powder can simply be mixed with hydrolyzed collagen powder. The powder can then be applied directly to a target (e.g., a hard surface, an implant, tissue, a wound, etc.). Alternately, the powder can be dissolved (e.g., within a syringe) in an aqueous solvent, then applied as appropriate.

In some embodiments, polylysine powder can be mixed with hydrolyzed collagen powder, then dissolved in an aqueous solvent, and then dried and formulated into a combined powder.

In some embodiments, the hydrolyzed collagen-based composition with polylysine provides preservative efficacy for the formulation before use. During treatment, the hydrolyzed collagen-based composition imparts antimicrobial activity to treat or prevent a microbial infection.

In some embodiments, the biocompatibility of the hydrolyzed collagen-based compositions described herein lends itself to assisting with cellular and tissue healing, as well as, protection from or treatment of infection.

In some embodiments, any of the antimicrobial hydrolyzed collagen-based compositions described herein can be mixed with or include polar liquids, such as alcohols and water, and applied to or within living tissue; or in, on, or surrounding a device (e.g., an implant device). In some embodiments, the hydrolyzed collagen-based composition can be added to a polar liquid. For example, when applied to living tissue, e.g., within a wound or surgical site, the polar liquid can be blood or lymphatic fluid.

In some embodiments, the antimicrobial hydrolyzed collagen-based compositions described herein can be aqueous compositions. As used herein, “aqueous” compositions include, but are not limited to, solutions in water with solubilized components, emulsified solutions in water stabilized by surfactants or hydrophilic polymers, as well as, viscous or gelled homogeneous or emulsified solutions.

In some embodiments, the hydrolyzed collagen-based composition can be utilized in a powder state and can be placed into or on a tissue defects, wound, burn, or in, on, or surrounding a medical device (e.g., an implanted medical device or medical device prior to implantation). In some embodiments, the powder mixture can be hydrated by endogenous or exogenous fluid sources.

Examples of tissue defects (e.g., impaired tissue) that can be treated by the hydrolyzed collagen-based compositions described herein include, but are not limited to, surgical incision sites, lesions, fissures, fistulas and diverticula. These tissue defects can be physiological or the result of infection, surgery, cyst, tumor removal, or traumatic injury or remodeling of soft tissue or hard tissue, such as in skin and wound healing, plastic surgery, cosmetic surgery, reconstructive surgery, coating/sealing of skin replacement products, tendon repair, hernia repair, craniofacial surgery, ophthalmic surgery, cervicofacial rhytidectomy, abdominoplasty, breast augmentation, myocardium repair, cartilage repair, bone repair, joint repair, nerve repair, spinal cord repair, liver tissue regeneration, bladder repair, muscle repair, mastopexy, rheumatology, gynecomastia reduction, body contouring, skin rejuvenation, skin resurfacing, microsurgery, dermato-cosmetics for filling in wrinkles, masking scars or enhancing lips, and the like.

Examples of implanted medical devices referenced herein include, but are not limited to joint implants, bone repair implants (e.g., rods, screws, etc.), tissue implants, skin replacement products, heart and cardiovascular devices, pain control devices, neurological control devices, dental implants, and the like.

When the antimicrobial hydrolyzed collagen-based composition is applied to a biological substrate or medical device in either a hydrated or dry form, the mixture (based on weight percent solids) can contain polylysine at a concentration of 0.05 wt % to 50 wt %, or from 0.075 wt % to 25 wt %, from 0.1 wt % to 10 wt %, from 0.1 wt % to 5 wt %, from 0.1 wt % to 2 wt %, or from 0.1 wt % to 1 wt %. The mixture (based on weight percent solids) can contain hydrolyzed collagen at a concentration of 50 wt % to 99.95 wt %, or from 75 wt % to 99.925 wt %, or from 90 wt % to 99.9 wt %, or from 95 wt % to 99.9 wt %, or from 98 wt % to 99.9 wt %, or from 99 wt % to 99.9 wt %.

In some embodiments, the hydrolyzed collagen-based composition can include a hydrophobic vicinal diol as a further antimicrobial component. In some embodiments, the hydrophobic vicinal diol can be a monoalkyl glycol, a glycerol alkyl ether, a monoacyl glycerol, or a combination thereof. In some embodiments, the hydrophobic vicinal diol is present at a concentration of from 0.5 wt % to 20 wt %, or from 1 wt % to 18 wt %, or from 3 wt % to 15 wt % based on solids in the composition.

In some embodiments, the hydrolyzed collagen-based composition comprises at least one antimicrobial hydrophobic monoalkyl glycol, a hydrophobic glycerol alkyl ether, and a hydrophobic monoacyl glycerol. In addition to being branched or unbranched, these compounds can either be saturated or unsaturated.

In some embodiments, the monoalkyl glycol comprises at least one of caprylyl glycol (also known as SC-10®, 1,2-dihydroxyoctane, 1,2-octanediol, and 1,2-octylene glycol), propylene glycol, hexylene glycol, 2-methyl-2,4-pentanediol, 1,3-butylene glycol, triethylene glycol, glycol bis(hydroxyethyl) ether. In some embodiments, the monoalkyl glycols are selected from caprylyl glycol (1,2-dihydroxyoctane), propylene glycol, and propylene glycol. In some embodiments, the monoalkyl glycols include caprylyl glycol, a component of Sensiva® SC 10. In some embodiments, the monoalkyl glycols include glycerol 1-(2-ethylhexyl) ether (2-ethylhexylglycerin).

Sensiva® SC 10 is reported to combine the excellent skin care and deodorizing properties of 2-ethylhexylglycerin (Sensiva® SC 50) with the moisturizing and antimicrobial properties of caprylyl glycol. Additionally, Sensiva® SC 10 can contribute to the antimicrobial stability of cosmetic formulations. It can also be used to improve the efficacy of traditional cosmetic preservatives, such as parabens or phenoxyethanol (Schülke & Mayr, Sensiva® SC 10 Multifunctional Cosmetic Ingredient). Screening tests with Sensiva® SC 10 have shown that it reliably inhibits the growth and multiplication of Gram-positive odor causing bacteria, while at the same time it does not affect beneficial skin flora.

In some embodiments, Sensiva® SC 10 (caprylyl glycol) by Schülke & Mayr may be present in an amount of 0.05 to 5 wt-% based on solids in the composition. In some embodiments, Sensiva® SC 10 may be present in an amount of 0.1 to 4 wt-% based on solids in the composition.

In some embodiments, the hydrophobic glycerol alkyl ether includes at least one of 1-O-heptylglycerol, 1-O-octylglycerol, 1-O-nonylglycerol, 1-O-decylglycerol, 1-O-undecylglycerol, 1-O-dodecylglycerol, 1-O-tridecylglycerol, 1-O-tetradecylglycerol, 1-O-pentadecylglycerol, 1-O-hexadecylglycerol (chimyl alcohol), 1-O-heptadecylglycerol, 1-O-octadecylglycerol (batyl alcohol), 1-O-octadec-9-enyl glycerol (selachyl alcohol), glycerol 1-(2-ethylhexyl) ether (also known as octoxyglycerin, 2-ethylhexyl glycerin, 3-(2-ethylhexyloxy)propane-1,2-diol, and Sensiva® SC 50 (2-ethylhexylglycerin), 2-ethylhexyl diglycol ether, 2-ethylhexyl oligoglycol ethers, glycerol 1-heptyl ether, glycerol 1-octyl ether, glycerol 1-decyl ether, and glycerol 1-dodecyl ether, glycerol 1-tridecyl ether, glycerol 1-tetradecyl ether, glycerol 1-pentadecyl ether, glycerol 1-hexadecyl ether, or glycerol 1-octadecyl ether. In some embodiments, the hydrophobic glycerol alkyl ethers is selected from glycerol 1-(2-ethylhexyl) ether, (Sensiva® SC 50) and 1-O-dodecylglycerol, In some embodiments, the hydrophobic glycerol alkyl ethers is glycerol 1-(2-ethylhexyl) ether.

Sensiva® SC 50 reliably inhibits the Gram-positive odor-causing bacteria on the skin and is used in deodorant formulations. It is reported to boost the efficacy of traditional preservatives. In some embodiments, Sensiva® SC 50 (2-ethylhexylglycerin) by Schülke & Mayr may be present in an amount of 0.5 to 15 wt-% based on solids in the composition. In some embodiments, Sensiva® SC 50 may be present in an amount of 3 to 12 wt-% based on solids in the composition.

In some embodiments, the hydrophobic monoacyl glycerol includes at least one of 1-O-decanoylglycerol (monocaprin), 1-O-undecanoylglycerol, 1-O-undecenoylglycerol, 1-O-dodecanoylglycerol (monolaurin, also called glycerol monolaurate and Lauricidin®), 1-O-tridecanoylglycerol, 1-O-tetradecanoylglycerol (monomyristin), 1-O-pentadecanoylglycerol, 1-O-hexadecanoylglycerol, 1-O-heptadecanoylglycerol, and 1-O-octanoylglycerol (monocaprylin). In some embodiments, the hydrophobic monoacyl glycerol is selected from 1-O-decanoylglycerol, 1-O-dodecanoylglycerol, 1-O-tetradecanoylglycerol, and 1-O-octanoylglycerol. In some embodiments, hydrophobic monoacyl glycerolsare is 1-O-dodecanoylglycerol. Glycerols substituted in the 1-O-position are more preferred than those substituted in the 2-O-position, or disubstituted in the 1-O and 2-O positions.

Since the antimicrobial hydrophobic monoalkyl vicinal diol and an antimicrobial hydrophobic monoalkyl and monoacyl glycerol have hydrophilic —OH groups but low or negligible water solubility, in some embodiments a surfactant can be added to aid in solution compatibilization and homogeneity of these compounds.

The antimicrobial hydrolyzed collagen-based composition can include a metal chelating agent, a surfactant, or both. Where the hydrolyzed collagen-based composition is an aqueous solution, a water-soluble polymer can be added to increase solution viscosity, to change rheology, and/or to prolong residence time of the antimicrobial composition on a biological surface or medical device.

Chelating agents can enhance the susceptibility of bacteria and other organisms to the biocidal effects of the antimicrobial agent. Thus, a hydrolyzed collagen-based composition containing a chelating agent can be more effective in combating infection than one without a chelating agent. Additionally, chelating agents deactivate matrix metalloproteases (MMPs), enzymes that can impede tissue formation and healing by breaking down collagen. MMPs are often found at elevated levels in impaired tissue. Chelating agents bind to zinc ions, which are necessary for MMP activity, disrupting the MMP, causing deactivation, and thus facilitating healing.

In some embodiments, the chelating agent is selected from any compound that is suitable for medical or veterinary use and is able to sequester monovalent or polyvalent metal ions. Examples include, but are not limited to, sodium, lithium, rubidium, cesium, calcium, magnesium, barium, cerium, cobalt, copper, iron, manganese, nickel, strontium or zinc. The outermost surface of bacterial cells universally carries a net negative charge, which is usually stabilized by divalent cations such as Mg²⁺ and Ca²⁺. This is associated with the teichoic acid and polysaccharide elements of Gram-positive bacteria, the lipopolysaccharide of Gram-negative bacteria, and the cytoplasmic membrane itself. Thus, the chelating agent aids in destabilizing microorganisms. Additionally, the chelating agent may deactivate matrix metalloproteases, such as in inflammatory wounds, facilitating collagen development.

Suitable chelating agents comprise, but are not limited to, aminocarboxylic acids, citric acid and its salts, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N-bis(2-hydroxybenzyl)ethylenediamine-N,N-diacetic acid, ethylenediamine-N,N-diacetic acid, ethylenediamine-N,N-dipropionic acid, triethylenetetraaminehexaacetic acid, ethylenediamine-N,N′-bis(methylenephosphonic acid), iminodiacetic acid, N,N-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates such as editronate, and salts thereof. Suitable chelating agents include, for example but are not limited to, hydroxyalkylphosphonates as disclosed in U.S. Pat. No. 5,858,937, specifically the tetrasodium salt of 1-hydroxyethylidene-1,1-diphosphonic acid, also referred to as tetrasodium etidronate, commercially available from Monsanto Company as DeQuest 2016 diphosphonic acid sodium salt or phosphonate.

Especially preferred chelating agents are mixed salts of EDTA such as disodium, trisodium, tetrasodium, dipotassium, tripotassium, tetrapotasssium, lithium, dilithium, ammonium, diammonium, triammonium, tetraammonium, calcium and calcium-disodium,. In some embodiments, the chelating agent can be or include a disodium, trisodium or tetrasodium salt of EDTA. In some embodiments, the chelating agent can be or include disodium EDTA and trisodium EDTA.

Suitable surfactants include, but are not limited to, cationic, anionic, nonionic, amphoteric and ampholytic surfactants. Preferred surfactants are nonionic and amphoteric surfactants. The surfactants can have an HLB (hydrophilic-lipophilic balance) value of 18-30 in order to maintain the biocidal activity of the antimicrobial agents, which facilitating a non-cytotoxic composition. The surfactant lowers surface tension, facilitating wetting of a surface for enhanced activity of the biocidal agent and for assistance with debridement.

Suitable nonionic surfactants include ethylene oxide/propylene oxide block copolymers of poloxamers, reverse poloxamers, poloxamines, and reverse poloxamines. In some embodiments, poloxamers and poloxamines are preferred, with poloxamers generally being most preferred. Poloxamers and poloxamines are available from BASF Corp. under the trade names of Pluronic® and Tetronic®.

Suitable Pluronic surfactants include but are not limited to Pluronic F38 having a HLB of 31 and average molecular weight (AMW) of 4,700, Pluronic F68 having a HLB of 29 and AMW of 8,400, Pluronic 68LF having a HLB of 26 and AMW or 7,700, Pluronic F77 having a HLB of 25 and AMW of 6,600, Pluronic F87 having a HLB of 24 and AMW of 7,700, Pluronic F88 having a HLB of 28 and AMW or 11,400, Pluronic F98 having a HLB of 28 and AMW of 13,000, Pluronic F108 having a HLB of 27 and AMW of 14,600, Pluronic F127 (also known as Poloxamer 407) having a HLB of 18-23 and AMW of 12,600, and Pluronic L35 having a HLB of 19 and AMW of 1,900.

Another class of surfactant is that of the diamino block copolymers of ethylene oxide and propylene oxide sold under the trade name Tetronic®. An exemplary surfactant of this type is Tetronic 1107 (also known as Poloxamine 1107).

In addition to the above, other surfactants may be added, such as for example polyethylene glycol esters of fatty acids, e.g., coconut, polysorbate, polyoxyethylene or polyoxypropylene ethers of higher alkanes (C12-C18), polysorbate 20 available under the trademark Tween 20, polyoxyethylene (23) lauryl ether available under the trademark Brij 35, polyoxyethylene (40) stearate available under the trademark Myrj 52, and polyoxyethylene (25) propylene glycol stearate available under the trademark Atlas G 2612. Other neutral surfactants include nonylphenol ethoxylates such as nonylphenol ethoxylates, Triton X-100, Brij surfactants of polyoxyethylene vegetable-based fatty ethers, Tween 80, decyl glucoside, and lauryl glucoside.

Amphoteric surfactants suitable for use in antimicrobial hydrolyzed collagen-based compositions described herein can include materials of the type offered commercially under the trademark Miranol. Another useful class of amphoteric surfactants is exemplified by cocoamidopropyl betaine, commercially available from various sources.

Emollients/moisturizers and humectants can be added to the hydrolyzed collagen-based compositions to provide a more soothing antimicrobial composition when used topically. Emollients/moisturizers function by forming an oily layer on the top of the skin that traps water in the skin. Petrolatum, lanolin, mineral oil, dimethicone, and siloxy compounds are common emollients. Other emollients include isopropyl palmitate, isopropyl myristate, isopropyl isostearate, isostearyl isostearate, diisopropyl sebacate, propylene dipelargonate, 2-ethylhexyl isononoate, 2-ethylhexyl stearate, cetyl lactate, lauryl lactate, isopropyl lanolate, 2-ethylhexyl salicylate, cetyl myristate, oleyl myristate, oleyl stearate, oleyl oleate, hexyl laurate, and isohexyl laurate, lanolin, olive oil, cocoa butter, shea butter, octyldodecanol, hexyldecanol, dicaprylyl ether and decyl oleate.

Humectants include glycerin, lecithin, 1,2-propylene glycol, dipropylene glycol, polyethylene glycol, 1,3-butylene glycol, and 1,2,6-hexanetriol. Humectants function by drawing water into the outer layer of skin.

Anti-inflammatory agents can also be added, such as water-soluble derivatives of aspirin, vitamin C, methylsulfonylmethane, tea tree oil, and non-steroidal anti-inflammatory drugs.

It is often desirable to include water-soluble viscosity builders in the hydrolyzed collagen-based compositions described herein., particularly when the hydrolyzed collagen-based composition include (or will hydrate to include) an aqueous phase. Because of their demulcent effect and possible hydrophobic interactions with biological tissue, water-soluble polymers have a tendency to enhance the interaction with a biological tissue by means of a hydrated film on the surface. Because of this behavior, such water-soluble polymers can increase the residence time of the antimicrobial composition or gel on a biological tissue. Aqueous media may be incorporated into the hydrolyzed collagen-based composition or may be derived from a treatment surface that is wet with, for example, wound exudate, blood, or plasma.

Water-soluble viscosity builders useful herein include, but are not limited to, methylcellulose, hydroxyethylcellulose, hydroxypropylcellulose, hydroxypropylmethylcellulose, carboxymethylcellulose, polyquaternium-1, polyquaternium-6, polyquaternium-10, guar, hydroxypropylguar, hydroxypropylmethylguar, cationic guar, carboxymethylguar, hydroxymethylchitosan, hydroxypropylchitosan, carboxymethylchitosan, N-[(2-hydroxy-3-trimethylammonium)propyl]chitosan chloride, water-soluble chitosan, hyaluronic acid and its salts, chondroitin sulfate, heparin, dermatan sulfate, amylose, amylopectin, pectin, locust bean gum, alginate, dextran, carrageenan, xanthan gum, gellan gum, scleroglucan, schizophyllan, gum arabic, gum ghatti, gum karaya, gum tragacanth, pectins, starch and its modifications, tamarind gum, poly(vinyl alcohol), poly(ethylene oxide), poly(ethylene glycol), poly(methyl vinyl ether), polyacrylamide, poly(N,N-dimethylacrylamide), poly(N-vinylacetamide), poly(N-vinylformamide), poly(2-hydroxyethyl methacrylate), poly(glyceryl methacrylate), poly(N-vinylpyrrolidone), poly(dimethylaminoethyl methacrylate), poly(dimethylaminopropyl acrylamide), polyvinylamine, poly(N-isopropylacrylamide) and poly(N-vinylcaprolactam), the latter two hydrated below their Lower Critical Solution Temperatures, and the like, and combinations thereof.

If anionic hydrophilic polymers are utilized for enhancing viscosity, the overall polymer negative charge may electrostatically attract and accumulate the cationic polylysine and a greater concentration of polylysine will then be needed to provide biocidal efficacy comparable to the utilization of a neutral or cationic water-soluble polymer. Thus, preferred water soluble polymers are neutral or cationic in charge. Examples of neutral water soluble polymers include, but are not limited to, hydroxyethylcellulose, hydroxypropylcellulose, hydroxypropylmethylcellulose, guar, hydroxypropylguar, hydroxypropylmethylguar, poly(ethylene oxide), and poly(N-vinylpyrrolidone). Examples of cationic water soluble polymers include, but are not limited to, cationic chitosans, cationic cellulosics, and cationic guar. Chitosan polymers may also enhance the antimicrobial behavior of the antimicrobial composition. More preferred hydrophilic polymers comprise hydroxypropylmethylcellulose, hydroxypropylcellulose, hydroxypropylguar, hydroxymethylchitosan, poly(ethylene oxide), N-[(2-hydroxy-3-trimethylammonium)propyl]chitosan chloride, with hydroxymethylpropylcellulose being most preferred.

Essential oils can also be added to the hydrolyzed collagen-based composition as fragrance or aromatic agents, and/or as antimicrobial agents, including thymol, menthol, sandalwood, camphor, cardamom, cinnamon, jasmine, lavender, geranium, juniper, menthol, pine, lemon, rose, eucalyptus, clove, orange, mint, linalool, spearmint, peppermint, lemongrass, bergamot, citronella, cypress, nutmeg, spruce, tea tree, wintergreen (methyl salicylate), vanilla, and the like. More preferred essential oils include thymol, sandalwood oil, wintergreen oil and eucalyptol for antimicrobial properties and pine oil for fragrance.

The hydrolyzed collagen-based composition can also include wetting agents, buffers, gelling agents or emulsifiers. Other excipients could include various water-based buffers ranging in pH from 5.0-7.5, silicones, polyether copolymers, vegetable and plant fats and oils, vitamins, laurate esters, myristate esters, palmitate esters, and stearate esters.

One or more biologically active agents may be incorporated into the hydrolyzed collagen-based composition to provide a medical benefit to a living host. Examples of biologically active agents that can be incorporated into the hydrolyzed collagen-based composition include, but are not limited to, cells, stem cells, amniotic tissue, amniotic cells, growth factors, micronized decellularized skin tissue, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, manuka honey, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, transfection agents, vitamins, essential oils, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle. Such biologically active agents could be in either of the (R)-, (R, S)-, or (S)-configuration, or a combination thereof.

In some embodiments, the hydrolyzed collagen-based composition comprises at least one of cells, stem cells, amniotic tissue, amniotic cells, exosomes, growth factors, micronized decellularized tissue, granulated collagen, gelatin, or glycosaminoglycans. In some embodiments, the cells are animal cells. In some embodiments, the cells can be mammalian cells or non-mammalian cells.

In some embodiments, the hydrolyzed collagen-based composition comprises at least one additional ingredient selected from glycolipids, glycoproteins, immunological response modifiers, saccharides, and polysaccharides.

The hydrolyzed collagen-based composition may be delivered in a variety of forms. Exemplary forms include, but are not limited to, powders, films, sheets, liquids, suspensions, creams, pastes, foams, lotions, gels, sprays, aerosols, capsules and tablets. The hydrolyzed collagen-based composition can also be imbibed by swabs, cloth, sponges, foams, wound dressing materials and non-woven and paper products, such as paper towels and wipes. Formulations of the hydrolyzed collagen-based compositions described herein may additionally include organic solvents, emulsifiers, gelling agents, moisturizers, stabilizers, time release agents, dyes, and like components commonly employed in formulations for body administration.

The hydrolyzed collagen-based compositions described herein may also be applied to catheters, and other medical devices, in a hydrated or dried form to provide a coating that can prevent microbial attachment to the catheter, or other medical devices, when they are introduced to the body.

Alternatively, the hydrolyzed collagen-based composition described herein can be added to a solid or porous support and dried. Examples of porous supports include, but are not limited to, a polymeric foam, a polymer film, a woven, knitted or nonwoven material, and then applied directly to a tissue or medical device.

In this case the polymeric foam may also absorb tissue exudate, creating a hydrated environment for controlled release of hydrolyzed collagen, polylysine, or other additives.

In another aspect, a method of treating tissue comprising contacting the tissue with any hydrolyzed collagen-based composition described herein is provided.

In some embodiments, the tissue being treated is impaired tissue. In some embodiments, the impaired tissue comprises at least one of a cut, a wound, a lesion, a rash, a fistula, a burn, a void, a surgical site, diabetic foot ulcer, venous ulcer, pressure ulcer, tissue affected by cellulitis, dehisced wounds, necrotic wounds, traumatic wounds with foreign bodies (i.e., puncture wounds), or a medical implant site.

In some embodiments, the hydrolyzed collagen-based composition comprises a polylysine comprising ε-polylysine, α-polylysine, or a combination thereof. In some embodiments, the hydrolyzed collagen-based composition comprises ε-polylysine. In some embodiments, the hydrolyzed collagen-based composition comprises α-polylysine.

In some embodiments, the hydrolyzed collagen-based composition exhibits a kill rate of at least 1 log of planktonic microbes. In some embodiments, the hydrolyzed collagen-based composition exhibits a kill rate of at least 2 log, or at least 3 log, or at least 4 log, or at least 5 log of planktonic microbes.

In some embodiments, the hydrolyzed collagen-based composition enhances a natural tissue regeneration process.

### EXPERIMENTAL

The following materials and abbreviations are used in the experimental section.

| | Source | Trade Name | Lot |
|---|---|---|---|
| Hydrolyzed | Sanara MedTech | Cellerate | C521274 |
| collagen - A | | | |
| Hydrolyzed | Sanara MedTech | Cellerate | C521231 |
| collagen - B | | | |
| Hydrolyzed | Gelita | Peptiplus XB | 822174 |
| collagen - C | | | |
| Hydrolyzed | Sanara MedTech | Cellerate | C520253 |
| collagen - D | | | |
| Hydrolyzed | Gelita | Peptiplus XB | 8621390- |
| collagen - E | | | 082322 |
| ε- Polylysine | Handary | ε -Polylysine | 20120210904 |
| α-Polylysine | EMD Millipore | α-Poly-D- | 4120981 |
| | | lysine (1 mg/ | |
| | | mL in water) | |
| Monolaurin | Med-chem labs | N/A | 40129264 |
| EDTA-Disodium | JT Baker | N/A | H25593 |
| salt | | | |
| EDTA-Trisodium | Spectrum | N/A | 1DK0688 |
| salt | | | |
| SC-10 (1,2- | Schulke | Sensiva -10 | 1272204 |
| Dihydroxyoctane) | | | |
| SC-50 | Schulke | Sensiva - 50 | 1257726 |
| (Glycerol 1-(2- | | | |
| ethylhexyl)ether) | | | |
| NaCl | EMD | N/A | K39163704A |
| Gelatin | Gelita | Bovine Limed | 614901 |
| | | Bone Gel, | |
| | | 150 BL | |
| Pullulan | Best of Chemicals | 90-130 mm\hat{ }2/s | B21B10272 |
| | Sciences | | |
| Maltodextrin | Grain Processing | Maltrin QD M585 | 4120980 |
| | Corp | | |

The Gelita hydrolyzed collagen listed above is from bovine collagen and includes collagen type I, II, IV, V, VI, and XVII. In some embodiments, the hydrolyzed collagen is formed from collagen including at least collagen type I. In some embodiments, the hydrolyzed collagen is formed from collagen including at least collagen type II. In some embodiments, the hydrolyzed collagen is formed from collagen including at least collagen type IV. In some embodiments, the hydrolyzed collagen is formed from collagen including at least collagen type V. In some embodiments, the hydrolyzed collagen is formed from collagen including at least collagen type VI. In some embodiments, the hydrolyzed collagen is formed from collagen including at least collagen type XVII.

### Example 1: Hydrolyzed Collagen and Polylysine Antimicrobial Testing

Two formulations were prepared: (1) a powder including hydrolyzed collagen-A (99.9 wt %) with ε-polylysine (0.1 wt %) and (2) a powder containing hydrolyzed collagen-A (99 wt %) with ε-polylysine (1 wt %). Effectiveness of formulations was evaluated in a standard kill-rate test where the described formulations were dissolved in phosphate buffered saline followed by the addition of microbial inoculum. Antimicrobial efficacy against planktonic P. aeruginosa after 2-hours was tested. Both formulations produced complete kill of planktonic P. aeruginosa.

### Example 2: Hydrolyzed Collagen and Polylysine Anti-Biofilm Testing

Anti-biofilm testing was performed on pre-formed 24-h old biofilms of P. aeruginosa in a 96-well plate format with the formulations dissolved in phosphate buffered saline and total 200 μL/well test volume. The formulations of Example 1 were tested for efficacy against a 1-day old P. aeruginosa biofilm. The formulation with 0.1 wt % ε-polylysine produced a 4-log kill with 24-hour treatment. The formulation with 1 wt % ε-polylysine in phosphate buffered saline produced a 5-log reduction. Additionally, a formulation containing 1 wt % ε-polylysine and 1% monolaurin in phosphate buffered saline produced a 6-log reduction in the same experiment.

### Example 3: Hydrolyzed Collagen and ε-Polylysine with Other Antimicrobials

The four formulations shown in the chart below were tested for antimicrobial efficacy.

TABLE 1
Antimicrobial additives with hydrolyzed collagen and polylysine

| | Formu- | Formu- | Formu- | Formu- |
|---|---|---|---|---|
| | lation 1 | lation 2 | lation 3 | lation 4 |
| Component | wt % | wt % | wt % | wt % |
| Hydrolyzed | 98 | 98.705 | 99.9 | 99 |
| collagen-C | | | | |
| ε- Polylysine | 1 | 0.1 | 0.1 | 1 |
| Monolaurin | 1 | | | |
| EDTA-Disodium | | 0.05 | | |
| salt | | | | |
| EDTA-Trisodium | | 0.015 | | |
| salt | | | | |
| SC-10 | | 0.3 | | |
| SC-50 | | 0.1 | | |
| NaCl | | 0.73 | | |

All formulations produced complete kill of planktonic P. aeruginosa following 2-hour treatment.

### Example 4: Cell Viability and Proliferation

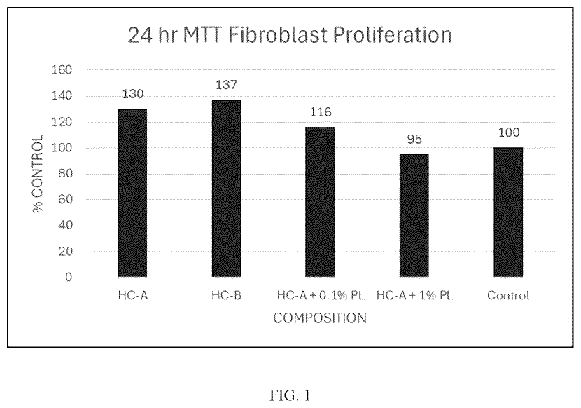

The formulations from Example 1 were compared to two lots of hydrolyzed collagen (A and B from materials list). As shown in FIG. 1, hydrolyzed collagen by itself (HC-A and HC-B) provided increased fibroblast viability and proliferation (130% and 137%, respectively) compared to the control (100%), while hydrolyzed collagen with 0.1 wt % ε-polylysine provided increased (116%) MTT values compared to the control, and hydrolyzed collagen with 1 wt % ε-polylysine provided equivalent (95%) MTT values compared to the control. Considering that the same formulations resulted in complete kill of P. aeruginosa, the selectivity index of the formulations is ≥10.

Primary human dermal fibroblasts (HDFa, PCS-201-012, P5) were cultured to 100% confluency in a T75 flask using fibroblast basal medium. The cells were detached and resuspended in 2% low serum medium containing penicillin, streptomycin, and amphotericin B to prevent infection. They were then seeded at N=3 for each formulation and control. Each well was charged with 0.5 mL of cell suspension. The plate was incubated for 24 hours at 37° C. The formulations were prepared at 1.75% w/v in serum-free fibroblast medium supplemented with antibiotics (penicillin, streptomycin, and amphotericin B). Once the media was removed, 1 mL of each solution was added to the seeded wells. The plates were then placed back in the incubator for 24 hours. The next day, MTT (3-(4,5-dimethylthiazol-2-yl)-2,5-diphenyltetrazolium bromide) solution was used, and 1 mL of this solution was added to each well. The plate was incubated for 4 hours at 37° C. After incubation, the medium was removed from each well, and 1 mL of solubilizing solution (dimethyl sulfoxide (DMSO)) was added to each well. Next, 200 μL from each well of the 24-well plate was transferred to a corresponding well in a 96-well plate. The plate reader then measured the absorbance of each well at 540 nm. The data were normalized using the 2% fibroblast basal media as the percentage control.

### Example 5: Antimicrobial Efficacy Comparison

Hydrolyzed collagen-D powder was mixed with polylysine powder (either α-polylysine, ε-polylysine, or both) to yield a total of 25 grams of powder; and stored for 24 hours at room temperature. Then, 1 gram of the mixture was placed in a sterile cup and 0.1 mL of bacteria/fungi (2×10⁷ CFUs) added; and stored for an additional 24 hours at 25° C. Then, 9 mL of Dey Engley (D/E) broth was added and mixed well. The compositions (0.1 mL) were placed in agar plates in triplicate at 37° C. for 24-48 hrs. Colonies were enumerated, and log reduction calculated. Phosphate buffered saline was the control and had no antimicrobial activity. Methicillin-resistant S. aureus (MRSA), P. aeruginosa, and C. albicans were the microbes used for this antimicrobial testing.

Hydrolyzed collagen by itself yielded 0.82 log reduction of C. albicans; 0.88 log reduction of P. aeruginosa; and 0.99 log reduction of MRSA. Hydrolyzed collagen mixed with either 0.1 wt % ε-polylysine or 1.0 wt % ε-polylysine yielded complete kill (e.g., log 6 kill) of C. albicans, P. aeruginosa, and MRSA. As discussed in Example 4, above, 1.0% ε-polylysine reduced fibroblasts by no more than 5%. Thus, the data demonstrates a selectivity index of greater than 10. In particular, 1.0% ε-polylysine killed ˜5% of fibroblast (so more would be required to reach 9000 kill) and 0.100 ε-polylysine provides complete kill (log 6 kill) of C. albicans, P. aeruginosa, and MRSA. Thus, the selectivity index is >1 divided by 0.10, which is greater than 10.

### Example 6: Hydrolyzed Collagen with Both α and ε-Polylysine

Hydrolyzed collagen-E and α and/or ε-polylysine were dissolved in deionized (DI) water to produce aqueous solutions. They were prepared at 65 wt solids (3.25 g solids with total weight of 5 g), which produced a gel (Table 2). Of note, the gels were all dilatant, shear thickening, and were suitable for medical device coatings as demonstrated by the gel coatings on silicone. α-Polylysine at 0.05 wt % (Sample 3) solubilized the hydrolyzed collagen gel to produce a clear liquid; whereas, ε-polylysine Sample A remained a slightly opaque gel similar to hydrolyzed collagen by itself at 65 wt % solids in water.

TABLE 2
Hydrolyzed collagen with α and ε-polylysine

| | | | wt % | wt % | Ave | |
|---|---|---|---|---|---|---|
| Sample | Chemical | Grams | Gel | Solids | pH | Observations |
| A | Hydrolyzed | 3.2489 | 64.58 | 99.94 | 6.05 | Color = Yellow, slightly opaque |
| | collagen E | | | | | Flow = slowly flowable gel, moves up spatula |
| | ε- | 0.0018 | 0.04 | 0.06 | | when mixing; dilatant |
| | Polylysine | | | | | Silicone Tube Coating = uniform coat, no |
| | Water | 1.7804 | 35.39 | N/A | | dripping, slight tack (non-stringy) |
| B | Hydrolyzed | 1.6252 | 32.49 | 49.99 | 5.57 | Color = amber, clear |
| | collagen E | | | | | Flow = flowable gel, moves up spatula when |
| | ε- | 1.6259 | 32.50 | 50.01 | | mixing; dilatant |
| | Polylysine | | | | | Silicone Tube Coating = uniform coat, no |
| | Water | 1.7518 | 35.02 | N/A | | dripping, slight tack (non-stringy) |
| C | Hydrolyzed | 3.2490 | 64.95 | 66.99 | 6.10 | Color = yellow, clear |
| | collagen E | | | | | Flow = slowly flowable gel, moves up spatula |
| | α- | 1.6010 | 32.00 | 33.01 | | when mixing; dilatant |
| | Polylysine | | | | | Silicone Tube Coating = uniform coat, no |
| | Water | 0.1526 | 3.05 | N/A | | dripping, slight tack (non-stringy) |
| D | Hydrolyzed | 3.1850 | 63.70 | 66.54 | 6.19 | Color = yellow, clear |
| | collagen E | | | | | Flow = slowly flowable gel, moves up spatula |
| | α- | 1.6003 | 32.01 | 50.24 | | when mixing, dilatant |
| | Polylysine | | | | | Silicone Tube Coating = uniform coat, no |
| | ε- | 0.0016 | 0.03 | 0.03 | | dripping, slight tack (non-stringy) |
| | Polylysine | | | | | |
| | Water | 0.2131 | 4.26 | N/A | | |

### Example 7: Hydrolyzed Collagen with Polylysine with Protein and Polysaccharides

Hydrolyzed collagen powder, polylysine powder and other additive powders were blended together to yield 5 grams total of blended powder (Table 3). All of the samples were freely flowable and non-tacky.

TABLE 3
Hydrolyzed collagen with proteins and polysaccharides

| | | | wt % | |
|---|---|---|---|---|
| Sample | Chemical | Grams | Solids | Observations |
| E | Hydrolyzed | 0.5012 | 10.20% | Color = yellow |
| | collagen E | | | Freely flowable, non-cohesive, similar angle of repose, can |
| | Gelatin | 4.4959 | 89.87% | be compressed/tapped to occupy slightly smaller volume. |
| | Polylysine | 0.0054 | 0.11% | |
| F | Hydrolyzed | 4.4955 | 89.88% | Color = light yellow |
| | collagen E | | | Freely flowable, non-cohesive, similar angle of repose, can |
| | Gelatin | 0.5003 | 10.00% | be compressed/tapped to occupy slightly smaller volume. |
| | Polylysine | 0.0058 | 0.12% | |
| G | Hydrolyzed | 0.2510 | 5.02% | Color = yellow |
| | collagen E | | | Freely flowable, non-cohesive, similar angle of repose, can |
| | Gelatin | 4.7013 | 93.98% | be compressed/tapped to occupy slightly smaller volume. |
| | Polylysine | 0.0502 | 1.00% | |
| H | Hydrolyzed | 3.7012 | 74.00% | Color = very light yellow |
| | collagen E | | | Freely flowable, non-cohesive, similar angle of repose, can |
| | Pullulan | 1.2507 | 25.00% | be compressed/tapped to occupy slightly smaller volume. |
| | Polylysine | 0.0500 | 1.00% | |
| I | Hydrolyzed | 1.2515 | 25.02% | Color = very light yellow |
| | collagen E | | | Freely flowable, non-cohesive, similar angle of repose, can |
| | Maltodextrin | 3.7000 | 73.97% | be compressed/tapped to occupy slightly smaller volume. |
| | Polylysine | 0.0508 | 1.02% | |

### Example 8: Hydrolyzed Collagen and Polylysine Films

Two formulations were made containing water, hydrolyzed collagen-E, and ε-Polylysine. One contained 2 wt % ε-polylysine (Sample K), one contained 5 wt % ε-polylysine (Sample L), and the control (Sample J) contained no ε-polylysine. The compositions are shown below in Table 4.

TABLE 4
Hydrolyzed collagen with polylysine compositions for films

| Sample | Polylysine (g) | Peptiplus XB (g) | Water (g) |
|---|---|---|---|
| J | 0 | 9.0032 | 6.0006 |
| K | 0.1006 | 4.9145 | 0 |
| L | 0.2507 | 4.7662 | 0 |

Samples J, K, and L were transferred to weigh boats to produce thin films (0.5 gram/boat) and thick films (3 grams/boat). Incorporation of the ε-polylysine reduced the stringiness observed with hydrolyzed collagen at high concentration in water. Following air drying at room temperature for 2 days, the thin film samples were all brittle. At 72 hours the thick film containing only hydrolyzed collagen (sample J) was brittle. The thick films containing ε-polylysine (samples K and L) were flexible after 72 hours of drying. However, after 5 days of air drying at room temperature, all films containing ε-polylysine (samples K and L) became brittle indicating amount of hydration is linked to the performance and behavior of the films and can be used to tune the material properties based on the intended use/purpose.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents

What is claimed is:

1. A hydrolyzed collagen-based composition comprising hydrolyzed collagen and 0.01 to 50 wt-% of polylysine based on the percent solids of the hydrolyzed collagen-based composition.

2. The hydrolyzed collagen-based composition of claim 1, comprising 0.05 to 25 wt-% of polylysine based on the percent solids of the hydrolyzed collagen-based composition.

3. The hydrolyzed collagen-based composition of claim 1, comprising 0.1 to 2 wt-% of polylysine based on the percent solids of the hydrolyzed collagen-based composition.

4. The hydrolyzed collagen-based composition of claim 1, wherein the polylysine comprises ε-polylysine.

5. The hydrolyzed collagen-based composition of claim 1, wherein the polylysine comprises α-polylysine.

6. The hydrolyzed collagen-based composition of claim 1, wherein the polylysine comprises both ε-polylysine and α-polylysine.

7. The hydrolyzed collagen-based composition of claim 6, wherein a weight ratio of ε-polylysine to α-polylysine ranges from 1:5 to 5:1.

8. The hydrolyzed collagen-based composition of claim 1, wherein the composition exhibits a kill rate of at least 1 log reduction of planktonic microbes.

9. The hydrolyzed collagen-based composition of claim 1, wherein the composition enhances a natural tissue regeneration process.

10. The hydrolyzed collagen-based composition of claim 1, further comprising 0.1 to 5 wt-% of a laurate ester based on the percent solids of the composition.

11. The hydrolyzed collagen-based composition of claim 1, comprising a chelating agent selected from the group consisting of aminocarboxylic acids, citric acid and its salts, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N′-bis(2-hydroxybenzyl)ethylenediamine-N,N′-diacetic acid, ethylenediamine-N,N′-diacetic acid, ethylenediamine-N,N′-dipropionic acid, triethylenetetraaminehexaacetic acid, ethylenediamine-N,N′-bis(methylenephosphonic acid), iminodiacetic acid, N,N′-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates.

12. The hydrolyzed collagen-based composition of claim 1, further comprising a biologically active agent selected from cells, stem cells, amniotic tissue, amniotic cells, growth factors, micronized decellularized tissue, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, manuka honey, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, transfection agents, vitamins, essential oils, liposomes, exosomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle.

13. The hydrolyzed collagen-based composition of claim 1, further comprising at least one of cells, stem cells, amniotic tissue, amniotic cells, exosomes, growth factors, micronized decellularized tissue, granulated collagen, gelatin, or glycosaminoglycans.

14. The hydrolyzed collagen-based composition of claim 1, further comprising at least one additional ingredient selected from glycolipids, glycoproteins, immunological response modifiers, saccharides, and polysaccharides.

15. The hydrolyzed collagen-based composition of claim 1, wherein the composition has a form selected from a powder, a liquid, a gel, a paste, a cream, a suspension, an emulsion, a film, a sheet, a foam, a lotion, a spray, an aerosol, a capsule, or a tablet.

16. A method of treating tissue comprising contacting the tissue with a hydrolyzed collagen-based composition of claim 1.

17. The method of claim 16, wherein the tissue being treated is impaired tissue.

18. The method of claim 16, wherein the hydrolyzed collagen-based composition comprises a polylysine comprising ε-polylysine, α-polylysine, or a combination thereof.

19. The method of claim 16, wherein the hydrolyzed collagen-based composition exhibits a kill rate of at least 1 log of planktonic microbes.

20. The method of claim 16, wherein the hydrolyzed collagen-based composition enhances a natural tissue regeneration process.
